(19) United States
(12) Patent Application Publication
(10) Pub. No.: US 2020/0281533 A1
(43) Pub. Date: Sep. 10, 2020

(54) BIOLOGICAL INFORMATION OUTPUT DEVICE, BIOLOGICAL INFORMATION OUTPUT METHOD, AND NON-TRANSITORY COMPUTER READABLE MEDIUM

(71) Applicant: Shigetomo Mitani, Aichi (JP)

(72) Inventor: Shigetomo Mitani, Aichi (JP)

(73) Assignee: NIDEC MOBILITY CORPORATION, Aichi (JP)

(21) Appl. No.: 16/808,978

(22) Filed: Mar. 4, 2020

(30) Foreign Application Priority Data
Mar. 5, 2019 (JP) 2019-039822

(51) Int. Cl. A61B 5/00; A61B 5/11; A61B 5/0205; A61B 5/024

(52) U.S. Cl. CPC A61B 5/6893; A61B 5/725; A61B 5/11; A61B 5/0205; A61B 2503/22; A61B 5/02444; A61B 5/7221; A61B 5/7282; A61B 5/7203

(57) ABSTRACT

A biological information output device eliminates data having a value equal to or greater than a predetermined value from data obtained from an electromagnetic wave reflected on a body surface and acquires data having a value less than the predetermined value; specifies a frequency peak in a predetermined period of the acquired data; creates a histogram by adding an occurrence frequency at which the frequency peak appears for each different class as a frequency while defining a prescribed frequency range where the specified frequency peak exists as one class; and determines, when a frequency of a class having the frequency which is a maximum occurrence frequency among classes in the histogram exceeds a predetermined threshold value, the class as an initial value of biological information. The biological information output device extracts the biological information from the data based on a cycle specified by the determined initial value.

### CROSS-REFERENCES TO RELATED APPLICATIONS

This application is based upon and claims the benefit of priority from Japanese Patent Application No. 2019-039822, filed on Mar. 5, 2019; the entire contents of which are incorporated herein by reference.

### FIELD

One or more embodiments of the present invention relate to a biological information output device for outputting biological information specific to a living body such as a heartbeat rate.

### BACKGROUND

In the related art, a device for detecting, acquiring, and outputting biological information has been known.

For example, JP-A-2017-225559 describes that biological information is accurately detected by irradiating a living body with an electromagnetic wave and calculating an angular speed between an I signal and a Q signal based on a differential value between the I signal and the Q signal obtained from a reflected wave reflected from the living body.

JP-A-2017-225560 describes that an accurate output of biological information is enabled by determining that a large change has occurred on a body surface of a living body when an angular speed between an I signal and a Q signal obtained from a reflected wave of an electromagnetic wave from the living body exceeds a predetermined threshold value and stopping an output of the biological information.

### SUMMARY

However, in the technique described in JP-A-2017-225559, erroneous biological information may be output when there is a large movement of the body at the time of detection of the biological information.

In the technique described in JP-A-2017-225560, the output of erroneous biological information can be suppressed because the output of the biological information is stopped when there is a large movement of the body. However, in the case where the output of the biological information is stopped when the movement of the body is large, the output of the biological information is largely delayed until the living body is in a resting state.

Further, since some peaks appear periodically in the biological information, intervals of the peaks (cycle of heartbeat or the like) are measured in order to extract the biological information. When the movement of the body is large, a level of other minute noises becomes large, so that the peaks are buried in the noises, and extraction of the biological information becomes difficult.

An object of one aspect according to one or more embodiments of the present invention is to accurately output biological information and to avoid a large delay in the output of the biological information.

In order to solve the above problems, a biological information output device according to one or more embodiments of the present invention includes: an irradiation unit that irradiates a body surface of a living body with an electromagnetic wave; a reception unit that receives a reflected wave of the electromagnetic wave reflected on the body surface; a data acquisition unit that eliminates data having a value equal to or greater than a predetermined value from data obtained from the received reflected wave, and acquires data having a value less than the predetermined value; a peak specification unit that specifies a frequency peak in a predetermined period of the acquired data for each of a plurality of the predetermined periods; a histogram creation unit that creates a histogram by adding an occurrence frequency at which the frequency peak appears for each different class as a frequency while defining a prescribed frequency range where the specified frequency peak exists as one class; an initial value determination unit that determines, when a frequency of a class having the frequency which is a maximum occurrence frequency among classes in the histogram exceeds a predetermined threshold value, the class as an initial value of biological information; and a biological information extraction unit that extracts the biological information from the data based on a cycle specified by the determined initial value.

Further, in order to solve the above problems, a biological information output method according to one or more embodiments of the present invention includes: eliminating data having a value equal to or greater than a predetermined value from data obtained from a reflected wave of an electromagnetic wave reflected on a body surface of a living body, the body surface being irradiated with the electromagnetic wave, and acquiring data having a value less than the predetermined value; specifying a frequency peak in a predetermined period of the acquired data for each of a plurality of the predetermined periods; creating a histogram by adding an occurrence frequency at which the frequency peak appears for each different class as a frequency while defining a prescribed frequency range where the specified frequency peak exists as one class; determining, when a frequency of a class having the frequency which is a maximum occurrence frequency among classes in the histogram exceeds a predetermined threshold value, the class as an initial value of biological information; and extracting the biological information from the data based on a cycle specified by the determined initial value.

According to the above configuration, in a case where the initial value is determined, the data acquisition unit acquires data less than the predetermined value when the movement of the living body is small, and excludes data equal to or greater than the predetermined value when the movement of the living body is large. Thereby, even though a state in which the movement of the living body is intermittently large occurs, the determination processing of the initial value by the peak specification unit, histogram creation unit, and the initial value determination unit can be proceeded using the data acquired when the movement of the living body is small. Therefore, by obtaining an initial value with high accuracy, biological information can be extracted more accurately using the initial value.

A width of the class may be set wider so that a resolution of the initial value is lower than a resolution of extracting the biological information by the biological information extraction unit. In this way, it is possible to quickly specify the class of the frequency which is the maximum occurrence frequency by roughening the accuracy of the initial value by the initial value determination unit. Therefore, the determination processing of the initial value by the initial value determination unit can be quickly completed.

Further, according to the above configuration, the accuracy of the initial value becomes coarse by increasing the width of the class. Thereby, the class of the frequency which is the maximum occurrence frequency in the histogram is stabilized rather than narrowing the width of the class, so that the robustness against the influence of disturbance can be enhanced. In this manner, by increasing the width of the class and lowering the resolution of the initial value, the determination of the initial value can be stabilized.

The biological information extraction unit may perform filter processing with a pass bandwidth through which a frequency component specified by the initial value passes, in the data. The bandwidth is determined according to the resolution of the initial value.

According to the above configuration, the biological information of the frequency component according to the initial value can be extracted from the measurement data.

The biological information extraction unit may exclude, in the extracted biological information, the biological information in which a difference from the initial value is equal to or greater than a reference value from an output target.

According to the above configuration, when erroneous biological information that cannot be removed by the filter processing remains, the output of the biological information can be prevented.

The biological information extraction unit may extract the biological information from, in the data, the data having a cycle in which a difference from the cycle specified by the initial value is less than a reference value, and exclude the data having a cycle in which the difference from the cycle specified by the initial value is equal to or greater than the reference value, from a target of extracting the biological information.

According to the above configuration, the biological information can be extracted without performing the filter processing by comparing the data with the initial value.

The data acquisition unit may start the acquisition of the data in response to an event indicating a start of driving of a vehicle.

According to the above configuration, data is acquired from a person (living body) from the start of driving. Thereby, an initial value can be determined from the state at the time of driving, and biological information can be output based on the initial value.

The biological information output device according to one or more embodiments of the present invention may be implemented by a computer, and in this case, a biological information output program that causes the computer to implement the biological information output device by causing the computer to operate as each unit (software element) included in the biological information output device, and a computer-readable recording medium that records the program are also included in the scope of one or more embodiments of the present invention.

According to one or more embodiments of the present invention, it is possible to accurately output biological information and to avoid a large delay in the output of the biological information.

### BRIEF DESCRIPTION OF DRAWINGS

FIG. 1 is a diagram showing an installation of a biological information output device according to one embodiment of the present invention in a vehicle interior.

FIG. 2 is a block diagram showing a configuration of the above described biological information output device.

FIG. 3 illustrates: (a) a waveform diagram showing measurement data to be subjected to Fourier transform by an initialization processing unit in the biological information output device; (b) and (c) waveform diagrams showing Fourier transform signals obtained by Fourier transform of the measurement data; and (d) a diagram showing a histogram in which an appearance frequency of frequency peak of the Fourier transform signal is represented as a histogram for each range of a pulse rate.

FIG. 4 is a flowchart showing a processing procedure of initialization measurement and normal measurement by the biological information output device.

FIG. 5 is a waveform diagram showing operations of the initialization measurement and the normal measurement by the biological information output device.

FIG. 6 illustrates: (a) a waveform diagram showing measurement data from which a biological information extraction unit in the biological information output device extracts biological information; and (b) a waveform diagram showing bandpass filter processing by the biological information extraction unit based on an initial value determined by the initialization processing unit of the biological information output device.

### DETAILED DESCRIPTION

In embodiments of the invention, numerous specific details are set forth in order to provide a thorough understanding of the invention. However, it will be apparent to one of ordinary skill in the art that the invention may be practiced without these specific details. In other instances, well-known features have not been described in detail to avoid obscuring the invention.

### Embodiment 1

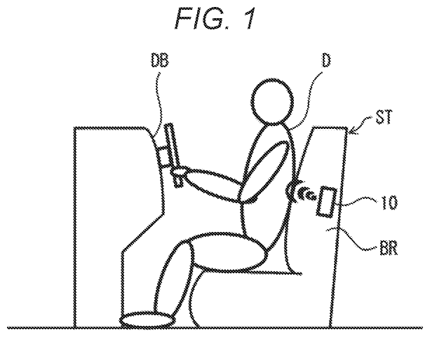

According to one embodiment of the present invention is described below with reference to FIGS. 1 to 6.

FIG. 1 is a diagram showing an installation of a biological information output device 10 in a vehicle interior according to an embodiment.

The biological information output device 10 shown in FIG. 1 is installed in, for example, an apparatus having a contact surface with which a part of a body of a human (human body) as a living body directly or indirectly contacts, and detects biological information of a user of the apparatus. The above-mentioned apparatuses are, for example, chairs, sofas, beds, physical examination apparatuses for medical institutions, seats (installed in vehicles, aircraft, ships, movie theaters, halls, or the like).

The contact surface corresponds to a seating surface or a backrest surface in a case of a chair or the like, and corresponds to a top surface of mattress in a case of a bed or the like. The contact surface may be directly or indirectly contacted with a part of a human body, and may be indirectly contacted in a clothed state. The part of the human body may be a hip or thigh in a case of a seating surface of a chair or the like, may be a back in general in a case of a backrest of a chair or the like or a bed or the like, and may be any of the limbs of the human body in a physical examination apparatus.

In the present embodiment, the biological information includes at least one of a heartbeat rate (pulse rate), a pulse wave size, a respiration rate, and a respiration size. Further, the biological information does not include coughing, sneezing, and the like that cause a movement of skin and muscles. The movement of skin and muscles is not derived from the above-described example(s) of the biological information.

The biological information output device 10 detects heartbeat or respiration that causes a minute movement on a body surface of a living body as biological information by the minute movement. In order to detect the minute movement, the biological information output device 10 irradiates the living body with electric waves, extracts biological information from reflected waves reflected from the living body, and outputs the extracted biological information.

In the present embodiment, as shown in FIG. 1, a case where the biological information output device 10 is installed in an interior of a vehicle will be described. The biological information output device 10 is disposed, for example, inside a backrest portion BR of a seat ST in which a driver D (living body) or the like sits.

As described above, the biological information output device 10 is intended to detect a minute movement of the skin surface caused by heartbeat or respiration. Therefore, it is preferable that the biological information output device 10 is disposed on the backrest portion BR having a contact surface with which the back of the driver D, where relatively little movement occurs, contacts, rather than being disposed on a dashboard DB in which the face or the like of the driver D, where a large movement occurs, is irradiated with electric waves from the front of the vehicle interior.

Next, the biological information output device 10 will be described in detail.

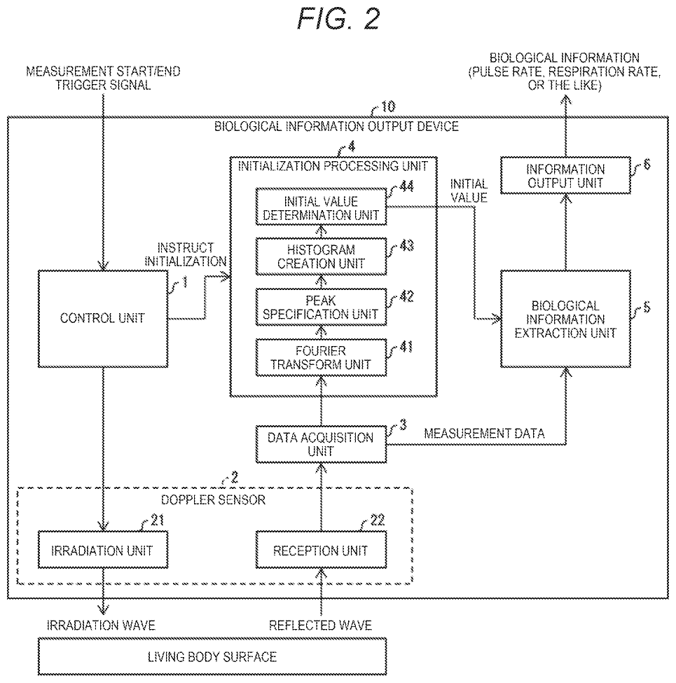

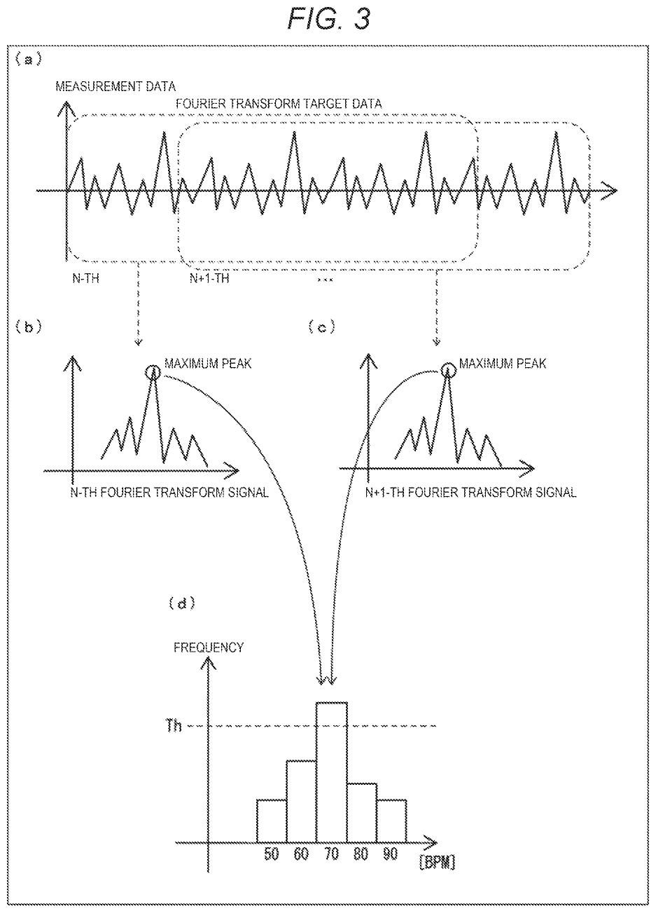

FIG. 2 is a block diagram showing a configuration of the biological information output device 10. The part (a) of FIG. 3 is a waveform diagram showing measurement data to be subjected to Fourier transform by an initialization processing unit in the biological information output device 10. The parts (b) and (c) of FIG. 3 are waveform diagrams showing Fourier transform signals obtained by Fourier transform of the measurement data. The part (d) of FIG. 3 is a diagram showing a histogram in which an appearance frequency of frequency peak of a Fourier transform signal is represented as a histogram for each range of a pulse rate.

As shown in FIG. 2, the biological information output device 10 includes a control unit 1, a Doppler sensor 2, a data acquisition unit 3, an initialization processing unit 4, a biological information extraction unit 5, and an information output unit 6.

The control unit 1 controls the Doppler sensor 2 and the initialization processing unit 4. When a measurement start trigger signal is input as a control of the initialization processing unit 4, the control unit 1 instructs the initialization processing unit 4 to start an initialization measurement. As a control of the Doppler sensor 2, the control unit 1 instructs an irradiation unit 21 of the Doppler sensor 2 to perform an irradiation of electromagnetic wave when the measurement start trigger signal is input, and also instructs the irradiation unit 21 of the Doppler sensor 2 to stop the irradiation of electromagnetic wave when a measurement end trigger signal is input.

The measurement start trigger signal is a signal serving as a trigger for starting the initialization processing by the initialization processing unit 4. As the measurement start trigger signal, an ON signal of a seat belt switch, which can assume a timing almost close to a start of driving, is used. The measurement end trigger signal is a signal serving as a trigger for ending biological information extraction processing by the biological information extraction unit 5. As the measurement end trigger signal, an OFF signal of the seat belt switch, which can assume a timing almost close to an end of driving, is used. By using such a measurement start trigger signal and a measurement end trigger signal, measurement of biological information is performed during driving.

Note that in a case of detecting a leave-behind of living body of an infant or a pet in the vehicle after driving, the control unit 1 may control the biological information extraction unit 5 to operate when the OFF signal of the seat belt switch is input. The control unit 1 may instruct the irradiation unit 21 to stop irradiation of electromagnetic waves when an output OFF signal, which is generated when an information output unit 6 no longer outputs biological information (when a living body is no longer present in the vehicle), is input as a measurement end trigger signal. Thereby, the measurement of the biological information at the time of parking and stopping the vehicle is performed until the output of the biological information disappears, that is, until the leave-behind state disappears.

The Doppler sensor 2 includes the irradiation unit 21, and a reception unit 22. The irradiation unit 21 emits an electromagnetic wave and irradiates a body surface (living body surface) of a living body (driver D) as an irradiation wave. The reception unit 22 receives a reflected wave in which the electromagnetic wave irradiated from the irradiation unit 21 is reflected on a living body surface, performs wave detection, amplification, and the like, and also outputs an I signal obtained by multiplying the emitted electromagnetic wave signal by the received reflected wave signal, and a Q signal obtained by delaying the I signal by a predetermined phase.

A data acquisition unit 3 acquires measurement data (data) by performing predetermined processing on the I signal and the Q signal from the reception unit 22. The data acquisition unit 3 outputs the acquired measurement data to the initialization processing unit 4 and the biological information extraction unit 5. Specifically, the data acquisition unit 3 converts analog I and Q signals into digital signals, and performs predetermined arithmetic operation processing on the digital I and Q signals to obtain measurement data. The arithmetic operation processing includes, but is not limited to, processing for averaging the amplitudes and phases of the I signal and the Q signal (amplitude and phase of the movement of the living body surface), processing for comparing the data with a threshold value, and the like.

The data acquisition unit 3 acquires and outputs measurement data that is less than the predetermined value, and excludes measurement data that is equal to or greater than the predetermined value without acquiring the measurement data. The predetermined value is less than or equal to data obtained for a large movement of the living body surface, and is set to a value greater than data for a small movement of the living body surface including a movement of the living body surface due to a heartbeat or respiration of the living body.

The initialization processing unit 4 performs processing of determining an initial value based on the measurement data from the data acquisition unit 3. The initial value is a value serving as a reference for the biological information extraction unit 5 to extract biological information from the measurement data.

In order to determine an initial value, the initialization processing unit 4 includes a Fourier transform unit 41, a peak specification unit 42, a histogram creation unit 43, and an initial value determination unit 44.

The Fourier transform unit 41 performs Fourier transform on the measurement data of a predetermined period extracted from the measurement data from the data acquisition unit 3. The Fourier transform unit 41 outputs a Fourier transform signal including a plurality of peaks as a frequency spectrum of the amplitude value of the measurement data by the Fourier transform processing. As shown in the part (a) of FIG. 3, a predetermined period (a period surrounded by a broken line in the figure) in which the Fourier transform unit 41 performs Fourier transform is shifted so that adjacent ones overlap with each other in part.

The peak specification unit 42 specifies a frequency peak (maximum peak) from a plurality of peaks included in the Fourier transform signal that is Fourier transformed for a predetermined period by the Fourier transform unit 41 (for example, the parts (b) and (c) of FIG. 3).

The histogram creation unit 43 creates a histogram by taking the prescribed range of frequencies in which the frequency peaks specified by the peak specification unit 42 exist as one class and accumulating the occurrence frequencies at which the frequency peaks appear for each different class to obtain a frequency for each class. For example, when a heartbeat included in the measurement data is extracted as a heartbeat rate that is biological information, the heartbeat rate of 10 (BPM) is set to a prescribed range as shown in the part (d) of FIG. 3. More specifically, prescribed ranges prescribed as 50 to 59 [BPM], 60 to 69 [BPM], 70 to 79 [BPM], 80 to 89 [BPM], and 90 to 99 [BPM] are defined as each class.

Further, the width of the class is set wide so as to be lower than the resolution at which the biological information extraction unit 5 extracts biological information. For example, when the biological information extraction unit 5 extracts the heartbeat rate as biological information with a 1 BPM resolution the class of the heartbeat rate is set with a width wider than 1 BPM (as in the above example, set in units of 10 BPM).

Here, a class having a frequency which is the maximum occurrence frequency among the classes in the histogram created by the histogram creation unit 43 is referred to a maximum-frequency class. When the frequency of the maximum-frequency class exceeds a predetermined threshold value (for example, the threshold value Th shown in the part (d) of FIG. 3), the initial value determination unit 44 determines the maximum-frequency class as the initial value of the biological information.

The biological information extraction unit 5 includes a bandpass filter having a variable pass bandwidth for extracting desired biological information based on the above-mentioned initial value determined by the initialization processing unit 4. Specifically, the biological information extraction unit 5 performs filter processing by a bandpass filter so as to extract the biological information from the measurement data output by the data acquisition unit 3 based on a cycle of the biological information specified by the initial value. In other words, the biological information extraction unit 5 performs filter processing with the pass bandwidth through which a frequency component specified by the initial value is passed.

Further, the biological information extraction unit 5 excludes, in the extracted biological information, the biological information in which the difference from the initial value is equal to or greater than a reference value from an output target.

The information output unit 6 outputs the biological information extracted by the biological information extraction unit 5 to the outside of the biological information output device 10. The information output unit 6 may include an amplifier or the like for amplifying biological information in order to output biological information in a form suitable for a device at a later stage utilizing biological information as necessary.

Next, the operation of the biological information output device 10 configured as described above will be described.

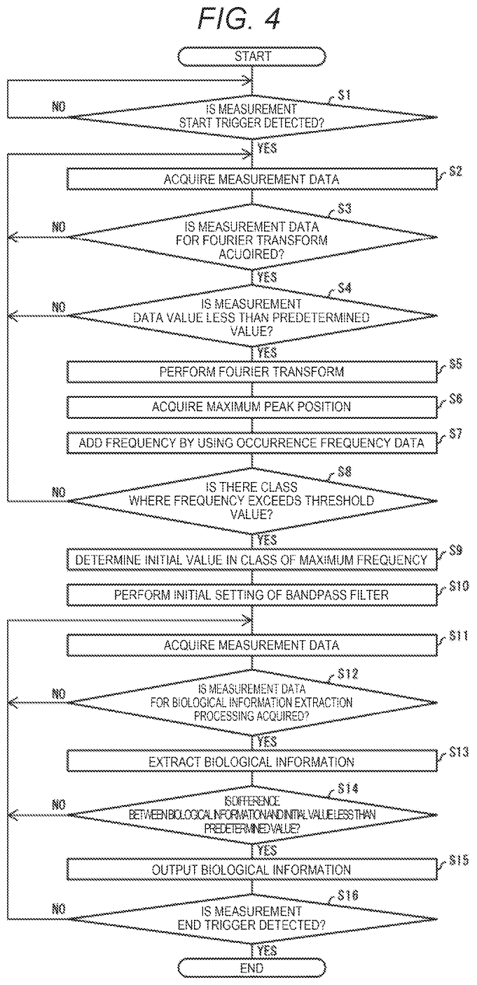

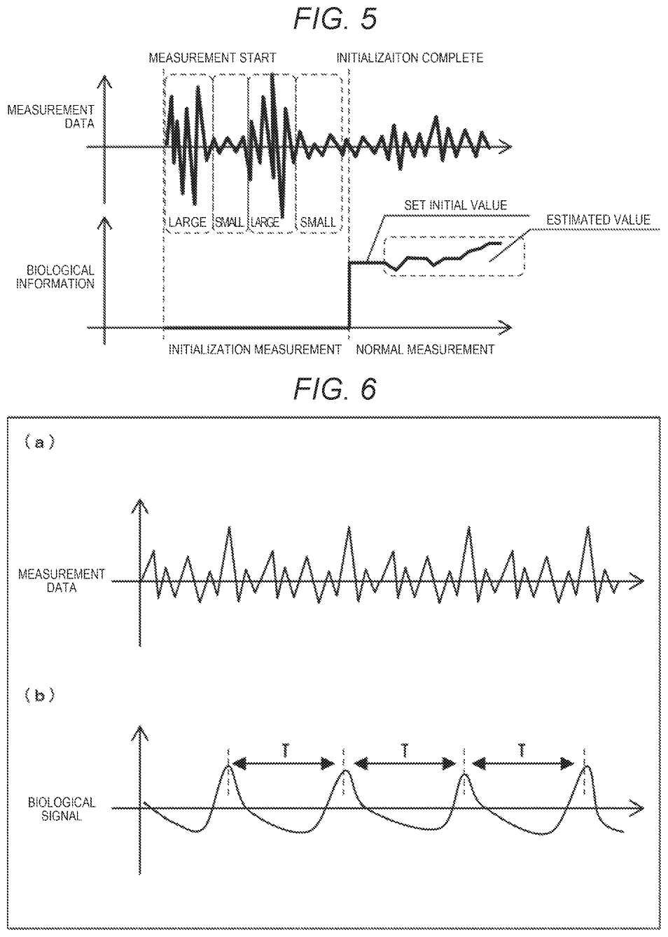

FIG. 4 is a flowchart showing a processing procedure of initialization measurement and normal measurement (biological information output method) by the biological information output device 10. FIG. 5 is a waveform diagram showing operations of the initialization measurement and the normal measurement by the biological information output device 10. The part (a) of FIG. 6 is a waveform diagram showing measurement data from which the biological information extraction unit 5 in the biological information output device 10 extracts biological information. The part (b) of FIG. 6 is a waveform diagram showing bandpass filter processing by the biological information extraction unit 5 based on the initial value determined by the initialization processing unit 4.

As shown in FIG. 4, first, the control unit 1 determines whether or not a measurement start trigger is detected (step S1). The control unit 1 waits until the measurement start trigger is detected (YES in step S1). When the measurement start trigger is detected, the control unit 1 instructs the irradiation unit 21 of the Doppler sensor 2 to perform an irradiation of an electromagnetic wave. As a result, when the irradiation unit 21 irradiates a living body surface with the electromagnetic wave, the reception unit 22 receives the reflected wave from the living body surface and outputs the detection signal in the form of, for example, an I signal and a Q signal.

Next, the data acquisition unit 3 acquires measurement data from the detected signal from the Doppler sensor 2 (step S2, data acquisition step). The data acquisition unit 3 determines whether or not the measurement data that can be subjected to Fourier transform by the Fourier transform unit 41 is acquired (step S3). In step S3, the data acquisition unit 3 determines, for example, whether or not the measurement data is abnormal data.

In step S3, when it is determined that the measurement data that can be subjected to the Fourier transform is not acquired (NO), the data acquisition unit 3 returns the processing to step S2 and acquires new measurement data. Further, in step S3, when it is determined that the measurement data that can be subjected to the Fourier transform is acquired (YES), the data acquisition unit 3 determines whether or not the value of the measurement data is less than a predetermined value (step S4).

In step S4, when it is determined that the value of the measurement data is not less than the predetermined value (the value is equal to or greater than the predetermined value) (NO), the data acquisition unit 3 returns the processing to step S2 and acquires new measurement data. Further, in step S4, when it is determined that the value of the measurement data is less than the predetermined value (YES), the data acquisition unit 3 outputs the measurement data. For example, as shown in FIG. 5, the data acquisition unit 3 discards the measurement data (shown as (large) in the figure) that is equal to or greater than the predetermined value so as to exclude the measurement data, and outputs the measurement data (shown as (small) in the figure) that is less than the predetermined value.

In the initialization processing unit 4, the Fourier transform unit 41 performs Fourier transform on the measurement data output from the data acquisition unit 3 (step S5). The Fourier transform unit 41 performs the Fourier transform using, for example, a predetermined period surrounded by a broken line in the part (a) of FIG. 3 as Fourier transform target data. In the examples shown in the parts (b) and (c) of FIG. 3, the Fourier transform unit 41 outputs an N-th Fourier transform signal and an N+1-th Fourier transform signal, respectively. In this manner, the Fourier transform unit 41 performs the Fourier transform on the measurement data of a large number of consecutive predetermined periods.

The part (a) of FIG. 3 shows the N-th and N+1-th Fourier transform target data. The predetermined period for acquiring the adjacent Fourier transform target data is partially overlapped in order to accumulate the frequency early. For example, in a case of acquiring 20 points (20 peaks) of measurement data per second, if it is necessary to collect 256 points (256 peaks) of measurement data for Fourier transform, it takes 10 seconds or more for this purpose. Therefore, if one frequency is obtained every 10 seconds, only two frequencies are obtained in 20 seconds.

To cope with such inconvenience, an overlapping period overlapping between adjacent predetermined periods is provided, and the measurement data acquired in the overlapping period between the previous predetermined period and the next predetermined period is used not only as the measurement data of the previous predetermined period but also as the measurement data of the next predetermined period. In this way, more measurement data can be collected for the Fourier transform in a short time.

The peak specification unit 42 specifies a peak relating to biological information from a plurality of peaks in the Fourier transform signal of a predetermined period converted from the measurement data by the Fourier transform unit 41, and acquires a position of the peak (step S6, peak specification step). When the biological information to be extracted is the pulse rate, the peak specification unit 42 specifies a position of the peak as a pulse rate. In the examples shown in the parts (b) and (c) of FIG. 3, the peak specification unit 42 specifies a position of the peak in the N-th Fourier transform signal and the N+l-th Fourier transform signal, respectively.

The peak specification method may be a maximum peak, or a plurality of peaks may be detected with about the top three peaks as candidates. Further, when a peak of a higher harmonic wave which is an integer multiple of the basic cycle of the biological information is included in the Fourier transform signal, the peak can be excluded from the target, and therefore the peak specification method is not limited to the above example.

The histogram creation unit 43 acquires a position of the maximum peak specified by the peak specification unit 42 as occurrence frequency data, and adds the frequency by using the acquired occurrence frequency data (step S7, histogram creation step). When the biological information to be extracted is a pulse rate, the histogram creation unit 43 adds the frequency “1” to the class to which the acquired occurrence frequency data applies. The histogram creation unit 43 creates a histogram by sequentially adding the frequency to a class to which the occurrence frequency data applies in this manner. The frequency is added every second by shifting the predetermined period for acquiring the Fourier transform target data described above, for example, by one second.

The initial value determination unit 44 determines whether or not there is a class where frequency exceeds the threshold value by the histogram creation unit 43 adding the frequencies (step S8). In step S8, when it is determined that there is no class where frequency exceeds the threshold value for determining the completion of initialization (NO), the initial value determination unit 44 returns the processing to step S2. Further, in step S8, when it is determined that there is a class where frequency exceeds the threshold value (YES), the initial value determination unit 44 determines an initial value in the class where frequency exceeds the threshold value (step S9, initial value determination step).

In the example shown in the part (d) of FIG. 3, the frequency included in a class with a pulse rate of 70 BPM (70 to 79 [BPM]) is most exceeding the threshold value Th. In this case, the initial value determination unit 44 determines a pulse rate of 75 [BPM], which is a median value of the class, as an initial value.

The biological information extraction unit 5 performs initial setting of the bandpass filter based on the initial value determined by the initial value determination unit 44 (step S10). For example, the biological information extraction unit 5 sets a pass bandwidth determined by an initial value which is determined from the measurement data as shown in the part (a) of FIG. 6 to a bandpass filter. Thereby, the bandpass filter of the biological information extraction unit 5 allows the biological signal (pulse wave data such as a pulse) of the cycle T shown in the part (b) of FIG. 6 to pass through from the input measurement data. When the initial value is 75 [BPM] as described above, the pass bandwidth of the bandpass filter is set to 1.32 to 1.17 [Hz] so as to allow the pulse wave data of 70 to 79 [BPM] to pass through.

When the above-described initial setting processing is completed, the processing shifts to normal measurement. The normal measurement can also be utilized to determine an abnormal state of a passenger during driving (a sudden change in illness or the like, a state of drowsiness or the like of the driver during driving) by comparing the data with a reference value. The normal measurement can also be useful for preventing malfunction of subsequent notification control by accurately determining that a living body such as an infant, a pet, or the like remains while a vehicle is stopped over about five minutes.

In the normal measurement, when the measurement data is acquired (step S11), the data acquisition unit 3 determines whether or not the measurement data that can be used for the biological information extraction processing is acquired (step S12). In step S12, when it is determined that the measurement data for the biological information extraction processing is not acquired (NO), the data acquisition unit 3 returns the processing to step S11. Further, in step S12, when the data acquisition unit 3 determines that the measurement data for the biological information extraction processing is acquired (YES), the biological information extraction unit 5 extracts the biological information based on the acquired measurement data (step S13, biological information extraction step).

Here, as shown in FIG. 5, in the initial stage of the normal measurement, the biological information is not extracted, and the set initial value is output.

The biological information extraction unit 5 determines whether or not a difference between the extracted biological information and the initial value is less than a reference value (step S14). In step S14, when it is determined that the difference between the biological information and the initial value is not less than the reference value (NO), the biological information extraction unit 5 excludes the biological information from the output target and returns the processing to step S11. Further, in step S14, when the difference between the biological information and the initial value is determined to be less than the reference value (YES), the biological information extraction unit 5 sets the biological information as an output target.

As the reference value, for example, the initial value is set. For example, when the initial value is 70 [BPM], the reference value is 70 [BPM]. Thereby, it is possible to exclude biological information that is twice or more the initial value (140 [BPM] or more). The reference value is not limited to this, and may be a value larger than the initial value, or may be a value smaller than the initial value.

The information output unit 6 outputs the biological information acquired as the output target from the biological information extraction unit 5 to the outside (step S15).

The control unit 1 determines whether or not the measurement end trigger signal is detected (step S16). In step S16, when it is determined that the measurement end trigger signal is not detected (NO), the control unit 1 returns the processing to step S11. Further, in step S16, when the control unit 1 determines that the measurement end trigger signal is detected (YES), the control unit 1 ends the biological information output processing.

Although the initialization measurement is performed over a certain period of time (for example, 40 frequencies are acquired in 40 seconds), in the normal measurement, the biological information is sequentially output every one second, for example. Further, the initialization measurement may also be performed intermittently or over a long period of time, such as 30 minutes. Thereby, it is possible to improve the accuracy of the initialization measurement.

As described above, the biological information output device 10 according to the present embodiment includes the control unit 1, the Doppler sensor 2, the data acquisition unit 3, the initialization processing unit 4, the biological information extraction unit 5, and the information output unit 6.

According to the above configuration, in a case where the initial value is determined, the data acquisition unit 3 acquires data less than the predetermined value when the movement of the living body is small, and excludes data equal to or greater than the predetermined value when the movement of the living body is large. Thereby, even though a state in which the movement of the living body is intermittently large occurs, the determination processing of the initial value by the initialization processing unit 4 can be proceeded using the data acquired when the movement of the living body is small. It is also possible to obtain an initial value with high accuracy. Therefore, biological information can be extracted more accurately using the initial value.

In this manner, the initial value is determined prior to extraction of the biological information by the biological information extraction unit. Therefore, it is possible to output more accurate biological information, and even when the movement of the living body is intermittently large, it is possible to greatly shorten the delay of the output of the biological information without stopping the output of the biological information until the state in which the movement of the living body is small is maintained continuously.

Further, the width of the class in the histogram created by the histogram creation unit 43 is set wide so as to be lower than the resolution at which the biological information extraction unit 5 extracts biological information.

According to the above configuration, the accuracy of the initial value becomes coarse by increasing the width of the class. Thereby, the class of the frequency which is the maximum occurrence frequency in the histogram is stabilized rather than narrowing the width of the class, so that the robustness against the influence of disturbance can be enhanced. In contrast to this, since the biological information such as the pulse includes the variation, it becomes difficult to obtain the maximum peak when the maximum peak is finely obtained for about one beat. On the other hand, in the extraction of the biological information, the biological information can be obtained with finer accuracy with reference to the roughly set initial value in order to grasp the change for each beat so as to follow the change of the biological information.

In this manner, by increasing the width of the class and increasing the resolution with which the initialization processing unit 4 determines the initial value, it is possible to stabilize the determination of the initial value. Therefore, more accurate extraction of biological information can be performed.

Further, the biological information extraction unit 5 performs filter processing with a pass bandwidth through which a frequency component specified by the initial value is passed, in the measurement data. The bandwidth is determined according to the resolution of the initial value.

Thereby, biological information of a frequency component according to the initial value can be extracted from the measurement data.

Further, the biological information extraction unit 5 excludes, in the extracted biological information, the biological information in which the difference from the initial value is equal to or greater than a reference value from an output target.

Thereby, when erroneous biological information that cannot be removed by the filter processing is extracted, the output of the erroneous biological information can be prevented.

Further, the data acquisition unit 3 starts acquisition of measurement data in response to an event indicating a start of driving of a vehicle. The event is an input or the like of the measurement start trigger signal described above (for example, an ON signal of a seat belt switch). Specifically, when the Doppler sensor 2 emits an electromagnetic wave and receives a reflected wave in response to an instruction given by the control unit 1 by inputting a measurement start trigger signal, the data acquisition unit 3 acquires measurement data.

Thereby, data is acquired from a person at the start of the driving. Therefore, it is possible to determine an initial value at the time of driving, and to output biological information based on the initial value.

### Embodiment 2

Another embodiment of the present invention is described below with reference to FIGS. 2 and 4. For the convenience of description, components having the same functions as those in the description of Embodiment 1 are denoted by the same reference numerals, and description thereof is omitted.

In the biological information output device 10 of Embodiment 1 described above, the biological information extraction unit 5 extracts biological information by a bandpass filter.

In contrast to this, as shown in FIG. 2, the biological information output device 10 of the present embodiment is basically configured in the same manner as the biological information output device 10 of Embodiment 1, but the biological information extraction unit 5 does not include a bandpass filter.

The biological information extraction unit 5 in the present embodiment extracts biological information from, in the measurement data, the measurement data having a cycle in which a difference from the cycle specified by the initial value is less than a reference value. Further, the biological information extraction unit 5 excludes, in the measurement data, the measurement data having the cycle equal to or greater than the reference value from a target of extraction of biological information.

The reference value is set in the same manner as the reference value to be compared with the biological information when the biological information extraction unit 5 in Embodiment 1 excludes erroneous biological information (the processing of step S14 in FIG. 4 described above). For example, the peak interval T in the part (b) of FIG. 6 is set to a reference value, and a peak interval in which a difference from the peak interval T is small is detected. The peak interval T serving as the reference value is determined in a cycle of taking the inverse of the frequency determined as the initial value.

The operation of the biological information output device 10 configured as described above will be described with reference to the flowchart in FIG. 4.

Similarly to the biological information output device 10 of Embodiment 1, the biological information output device 10 performs the processing of steps S1 to S9 to determine an initial value, but the processing of steps S10 to S16 is partially different as follows.

Specifically, since the biological information extraction unit 5 does not have a bandpass filter, the initial setting of the bandpass filter in step S10 is not performed. In the biological information extraction processing of step S13, as described above, the biological information extraction unit 5 extracts biological information from the measurement data having a cycle in which a difference from the cycle specified by the initial value is less than the reference value, and excludes the measurement data having a cycle in which the difference is equal to or greater than the reference value from the target of extraction of the biological information. Thereby, an interval for searching for the cycle of the biological signal is set based on the initial value. The biological information extraction unit 5 does not perform the processing of step S14.

The biological information output device 10 of the present embodiment can extract biological information without changing the pass band setting of the bandpass filter by providing the biological information extraction unit 5 configured as described above. Therefore, the simplification of processing can be implemented.

Implementation Example by Software

The control blocks (in particular, the initialization processing unit 4 and the biological information extraction unit 5) of the biological information output device 10 may be implemented by a logic circuit (hardware) formed on an integrated circuit (IC chip) or the like, or may be implemented by software.

In the latter case, the biological information output device 10 includes a computer that executes instructions of a program (biological information output program) which is software for implementing each function. The computer includes, for example, one or more processors and a computer-readable recording medium on which the program is recorded. In the computer, an object according to one or more embodiments of the present invention is achieved by the processor reading the program from the recording medium and executing the program. As the processor, for example, a central processing unit (CPU) can be used.

As the recording medium, tapes, disks, cards, semiconductor memories, programmable logic circuits, or the like can be used in addition to a “non-transitory tangible medium”, for example, read only memory (ROM), or the like. Further, a random access memory (RAM) or the like for developing the programs may be further provided.

The program may be supplied to the computer via any transmission medium capable of transmitting the program (a communication network or a broadcast wave or the like). Note that one embodiment of the present invention can also be implemented in the form of a data signal embedded in a carrier wave in which the program is embodied by electronic transmission.

### ADDITIONAL NOTES

According to one or more embodiments of the present invention, it is not limited to the above-mentioned embodiments, and various modifications can be made within the scope shown in the claims, and embodiments obtained by appropriately combining technical means disclosed in different embodiments are also included in the technical scope of the present invention.

1. A biological information output device comprising:
an irradiation unit that irradiates a body surface of a living body with an electromagnetic wave;
a reception unit that receives a reflected wave of the electromagnetic wave reflected on the body surface;
a data acquisition unit that eliminates data having a value equal to or greater than a predetermined value from data obtained from the received reflected wave, and acquires data having a value less than the predetermined value;
a peak specification unit that specifies a frequency peak in a predetermined period of the acquired data for each of a plurality of predetermined periods;
a histogram creation unit that creates a histogram by adding an occurrence frequency at which the frequency peak appears for each different class as a frequency while defining a prescribed frequency range where the specified frequency peak exists as one class;
an initial value determination unit that determines, when a frequency of a class having the frequency which is a maximum occurrence frequency among classes in the histogram exceeds a predetermined threshold value, the class as an initial value of biological information; and
a biological information extraction unit that extracts the biological information from the data based on a cycle specified by the determined initial value.

2. The biological information output device according to claim 1,
wherein a width of the class is set wider so that a resolution of the initial value is lower than a resolution of extracting the biological information by the biological information extraction unit.

3. The biological information output device according to claim 1,
wherein the biological information extraction unit performs filter processing with a pass bandwidth through which a frequency component specified by the initial value passes, in the data.

4. The biological information output device according to claim 3,
wherein the biological information extraction unit excludes, in the extracted biological information, the biological information in which a difference from the initial value is equal to or greater than a reference value from an output target.

5. The biological information output device according to claim 1,
wherein the biological information extraction unit extracts the biological information from, in the data, the data having a cycle in which a difference from the cycle specified by the initial value is less than a reference value, and excludes the data having a cycle in which the difference from the cycle specified by the initial value is equal to or greater than the reference value, from a target of extracting the biological information.

6. The biological information output device according to claim 1,
wherein the data acquisition unit starts acquisition of the data in response to an event indicating a start of driving of a vehicle.

7. A non-transitory computer readable medium storing a biological information output program, when executed by a computer, causing the computer to perform operations of the biological information output device according to claim 1.

8. A biological information output method comprising:
eliminating data having a value equal to or greater than a predetermined value from data obtained from a reflected wave of an electromagnetic wave reflected on a body surface of a living body, the body surface being irradiated with the electromagnetic wave, and acquiring data having a value less than the predetermined value;
specifying a frequency peak in a predetermined period of the acquired data for each of a plurality of predetermined periods;
creating a histogram by adding an occurrence frequency at which the frequency peak appears for each different class as a frequency while defining a prescribed frequency range where the specified frequency peak exists as one class;
determining, when a frequency of a class having the frequency which is a maximum occurrence frequency among classes in the histogram exceeds a predetermined threshold value, the class as an initial value of biological information; and
extracting the biological information from the data based on a cycle specified by the determined initial value.
